(19) United States
(12) Patent Application Publication
(10) Pub. No.: US 2025/0270510 A1
(43) Pub. Date: Aug. 28, 2025

(54) DEVICE AND METHOD FOR HIGH-THROUGHPUT PRODUCTION OF HYDROGELS

(71) Applicants: The Regents of the University of California, Oakland, CA (US); University of Colorado Boulder, Aurora, CO (US)

(72) Inventors: Brian Aguado, La Jolla, CA (US); Brandon Vogt, La Jolla, CA (US); Keith Koch, Aurora, CO (US)

(21) Appl. No.: 19/057,899

(22) Filed: Feb. 19, 2025

Related U.S. Application Data
(60) Provisional application No. 63/556,789, filed on Feb. 22, 2024.

(51) Int. Cl. C12N 5/0775; C12M 1/12

(52) U.S. Cl. CPC C12N 5/0668; C12M 25/04; C12N 2533/30; C12N 2537/10

(57) ABSTRACT

Disclosed are devices and methods that pertain to a high-throughput formation of flat hydrogels using a hydrogel stamping technique for simultaneously producing a plurality of hydrogels in a multi-well plate. In an embodiment of the disclosed technology, a hydrogel stamp device may include a base; a plurality of rods arranged on the base and configured to be inserted into a plurality of wells of a multi-well plate to enable formation of hydrogels inside the plurality of wells, wherein each of the plurality of rods extends vertically from the base and includes: a base end connected to the base; a hydrophobic end positioned opposite the base end; and a body portion between the base end and the hydrophobic end; and a spacer that surrounds at least part of the base ends of the plurality of rods.

### CROSS-REFERENCE TO RELATED APPLICATION

This application claims priority to U.S. Provisional Application No. 63/556,789, entitled “HIGH-THROUGHPUT PREPARATION OF FLAT HYDROGELS” and filed on Feb. 22, 2024. The entirety of the above application is incorporated by reference as part of the disclosure of this patent document.

### TECHNICAL FIELD

This patent document relates to the production of hydrogels.

### BACKGROUND

Hydrogels are three-dimensional polymer networks capable of absorbing and retaining large amounts of water while maintaining their structural integrity. These materials can be composed of natural, synthetic, or hybrid polymers and are widely used in biomedical, pharmaceutical, and industrial applications.

Among various hydrogel structures, flat hydrogels refer to hydrogels that are manufactured in sheet-like or planar forms. These flat hydrogels offer several advantages, including ease of handling, uniform thickness, and adaptability for applications requiring direct contact with biological tissues or surfaces.

Despite their numerous advantages, the production of flat hydrogels presents certain challenges. Traditional hydrogel fabrication techniques, such as bulk polymerization and mold-based methods, often result in inconsistent thickness, defects, or inefficient material usage. Additionally, achieving high throughput manufacturing while maintaining precise control over hydrogel properties, such as water content, mechanical strength, and surface smoothness, remains a significant challenge.

### SUMMARY

The technology disclosed in this patent document relates to devices and methods for the production of hydrogels.

In an embodiment of the disclosed technology, a hydrogel stamp device may include a base; a plurality of rods arranged on the base and configured to be inserted into a plurality of wells of a multi-well plate to enable formation of hydrogels inside the plurality of wells, wherein each of the plurality of rods extends vertically from the base and includes: a base end connected to the base; a hydrophobic end positioned opposite the base end; and a body portion between the base end and the hydrophobic end; and a spacer that surrounds at least part of the base ends of the plurality of rods.

In another embodiment of the disclosed technology, a method of simultaneously forming a plurality of hydrogels may include performing a vapor deposition process to apply a surface modifier onto inner surfaces of wells of a multi-well plate; dispensing a predetermined amount of a hydrogel precursor solution into each of the wells of the multi-well plate treated with the vapor deposition process; placing a hydrogel stamp device with a plurality of rods and a spacer, onto the multi-well plate such that each of the plurality of rods is inserted into a corresponding well of the multi-well plate; and performing a curing process such that the hydrogel precursor solution located between a hydrophobic end of each of the plurality of rods and the corresponding well of the multi-well plate is crosslinked.

### BRIEF DESCRIPTION OF THE DRAWINGS

FIG. 1A shows an example of a hydrogel stamp device that includes a stamp base and a plurality of glass rods. FIGS. 1B-1C show an example of a hydrogel stamp device that includes a stamp base, a plurality of glass rods, and a spacer. FIG. 1D shows an example of a multi-well plate.

FIGS. 2A-2D shows the results of experiments conducted for optimization of the gel stamping process.

FIG. 3A shows αSMA (Alpha-Smooth Muscle Actin) gradient mean intensity of male valvular interstitial cells (VICs) on soft gels. FIG. 3B shows αSMA gradient mean intensity of female VICs on soft gels. FIG. 3C shows αSMA gradient mean intensity of female adult rat ventricular fibroblasts (ARVFs) P3 (passage 3) on soft gels. FIG. 3D shows αSMA gradient mean intensity of female ARVFs P4 (passage 4) on soft gels.

FIG. 4A shows αSMA gradient mean intensity of male VICs on stiff gels. FIG. 4B shows αSMA gradient mean intensity of female VICs on stiff gels. FIG. 4C shows αSMA gradient mean intensity of female ARVFs P3 on stiff gels. FIG. 4D shows αSMA gradient mean intensity of female ARVFs P4 on stiff gels.

FIG. 5 shows an example method of simultaneously producing a plurality of hydrogels based on some embodiments of the disclosed technology.

### DETAILED DESCRIPTION

Section headings are used in the present document only for ease of understanding and do not limit scope of the embodiments to the section in which they are described.

The embodiments of the present application describe, among other features and benefits, a hydrogel stamp device and a high-throughput hydrogel fabrication method using the hydrogel stamp device and a multi-well plate. An example hydrogel stamp device includes a plurality of rods vertically attached to a base. The plurality of rods can fit into a plurality of wells of a multi-well plate and has a shape and size similar to the two-dimensional shape of the plurality of wells. The hydrogel stamp device is used to simultaneously produce a plurality of hydrogels by applying pressure simultaneously to the hydrogel precursor solution dispensed in all the wells of the multi-well plate. While maintaining this pressure, a curing process is performed. For example, while maintaining this pressure, ultraviolet (UV) curing is performed by irradiating UV light onto the hydrogel precursor solution located in each well between the bottom of the well and the end of the corresponding rod inserted into the well, enabling the simultaneous production of hydrogel with a thickness corresponding to the distance between the well bottom and the end of the rod. It is important that the hydrogel maintains its shape when the rod is removed from the well after the curing process. As further described below, applying a siliconizing agent to the end of the rod increases its hydrophobicity and by doing so, the end of the rod, which is in direct contact with the hydrogel formed inside the well, prevents the hydrogel from sticking to it. This ensures that when the rod is removed after the curing process, the hydrogel does not adhere to the rod end, preventing deformation of its shape. The hydrogel stamp device further includes a spacer that includes a plurality of holes configured to allow the plurality of rods to be inserted and pass through such that the spacer is placed on the base of the hydrogel stamp device to surround the bottom end of each of the plurality of rods. The spacer is detachable and replaceable. Different thicknesses of spacers can be used depending on the desired thickness of the hydrogel. The thickness of the spacer is determined based on: a height at which the plurality of rods protrudes above the base of the hydrogel stamp device, the depth of the plurality of wells in the multi-well plate, and the desired thickness of the hydrogels that will be produced in the plurality of wells.

A hydrogel stamp device implemented based on some embodiments of the disclosed technology can enable the simultaneous formation of a plurality of hydrogels (e.g., flat hydrogels) within a treated glass-bottom well plate, making them particularly suitable for cell culture applications and various immunoassays. In some embodiments of the disclosed technology, the hydrogel stamp device includes a combination of composite plastic and glass rods, which are hydrophobic enough to prevent adhesion to the hydrogels after curing (e.g., UV light). In some embodiments of the disclosed technology, the hydrogel stamp device may further include one or more interchangeable spacers of varying thicknesses, allowing for the formation of hydrogels with different heights. Additional details and examples of described in the sections that follow.

### Materials and Assembly of the Hydrogel Stamp Device

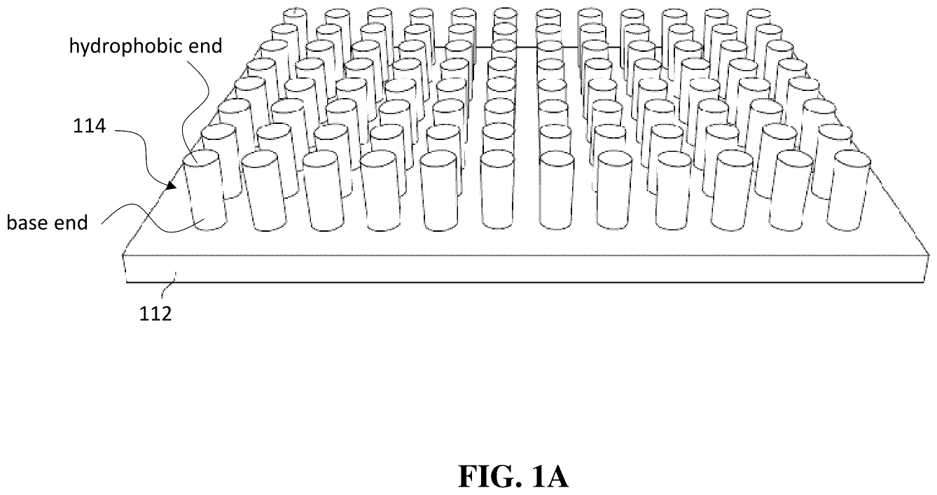

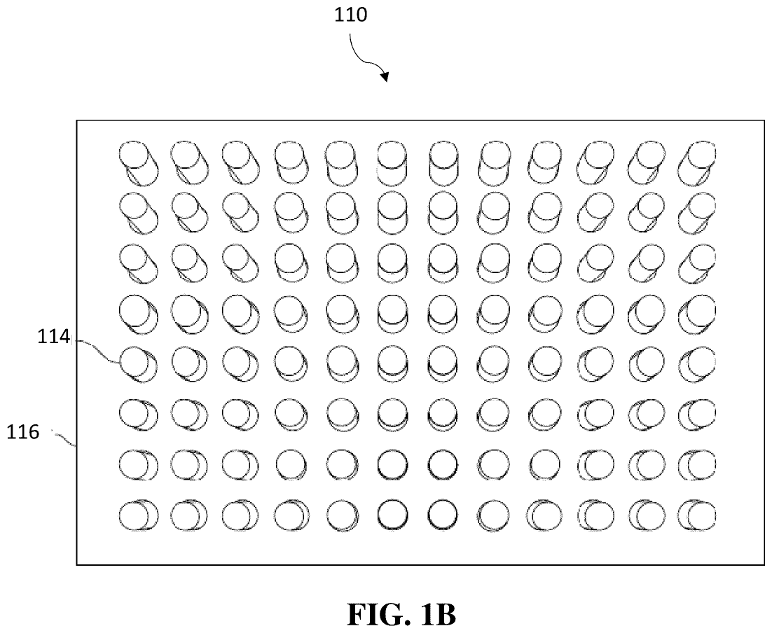

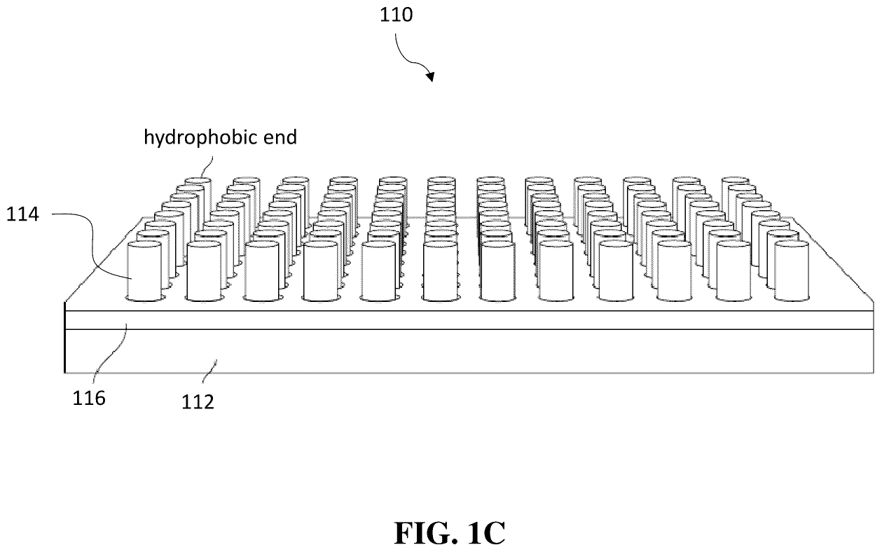

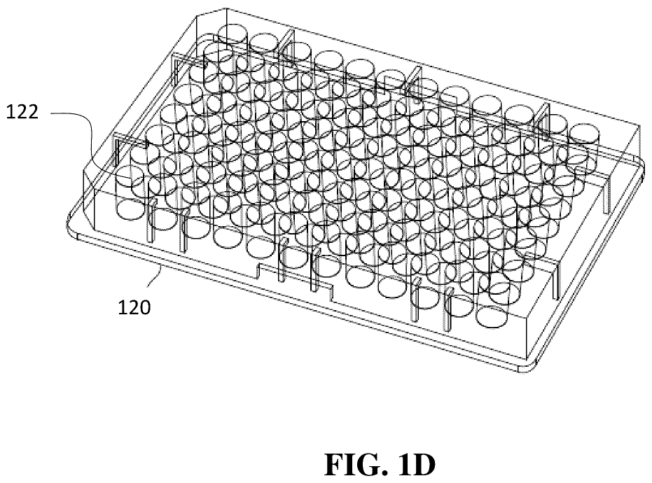

FIG. 1A shows an example of a hydrogel stamp device 110 that includes a stamp base 112 and a plurality of glass rods 114. FIGS. 1B-1C show an example of a hydrogel stamp device that includes a stamp base 112, a plurality of glass rods 114, and a spacer 116. FIG. 1D shows an example of a multi-well plate 120 that includes a plurality of wells 122.

In some embodiments of the disclosed technology, the hydrogel stamp device 110 includes a plurality of rods 114 with hydrophobic ends, a stamp base 112 configured to support the plurality of rods (e.g., glass rods) 114, and a hydrogel stamp device spacer 116 used to determine the final hydrogel height.

Hydrogel can be formed using the hydrogel stamp device 110 and the multi-well plate 120 in the following example method. A predetermined amount of a hydrogel precursor solution is dispensed into each of the wells 122 of the multi-well plate 120, and then the hydrogel stamp device 110 with the plurality of rods 114 and the spacer 116 is placed onto the multi-well plate 120 such that each of the plurality of rods 114 is inserted into a corresponding well 122 of the multi-well plate 120. The same pressure is simultaneously applied to the hydrogel precursor solution dispensed in all the wells 122 of the multi-well plate 120 while performing a curing process (e.g., exposing the hydrogel precursor solution to light of a specific wavelength such as ultra-violet light). The hydrogel precursor solution located between a hydrophobic end of each of the plurality of rods 114 and the corresponding well 122 of the multi-well plate 120 is simultaneously exposed to the ultra-violet light for crosslinking.

In some implementations, the rods 114 with hydrophobic ends may include glass rods. In some implementations, the stamp base 112 may include a plastic stamp base. In some embodiments of the disclosed technology, the shape and size of the rods (e.g., glass rods) 114 are determined based on the shape and size of the wells 124. For example, if the two-dimensional shape of the well is circular, the glass rod takes a cylindrical form, with its diameter designed to be smaller than that of the well. In some implementations, each of the plurality of rods may have a diameter of approximately 5 mm and a length of 21 mm. One end of each rod is mechanically polished to ensure a square surface finish with an angular tolerance of +0.5°, while the opposite end has a ground or machined finish. In some implementations, when a 96-well plate is used as the stamp base, the plurality of rods may include a total of 96 rods. In some implementations, the stamp base may include a plastic stamp base that is fabricated through three-dimensional (3D) printing.

In some embodiments of the disclosed technology, the hydrogel stamp device may also include spacers of varying thicknesses. In some implementations, the thicknesses of the spacers may include 0 mm, 0.25 mm, 0.5 mm, and 1 mm. In one example, the spacers may also be manufactured using 3D printing. In some implementations, the spacers may include detachable spacers.

To assemble the hydrogel stamp device, all required components are gathered and arranged on an even working surface. Each of a plurality of glass rods is then manually inserted into a plastic base, ensuring that the polished ends are oriented upward. Once all rods are in place, a temporary spacer is slid over them and fitted onto the plastic base. In some implementations, the temporary spacer may refer to a spacer that is used to align each glass rod properly with a corresponding well. As will be discussed below, the temporary spacer is removed, and the desired spacer is attached to the hydrogel stamp device to determine the final hydrogel height. The entire stamp assembly (e.g., hydrogel stamp device) is then inverted and pressed into a multi-well plate such as 96-well plate (e.g., 96-well glass bottom plate), ensuring that each glass rod properly aligns with a corresponding well. In some implementations, a 96-well plate is a flat plate with 96 wells that are used to hold samples, reagents, or substances. In some implementations, examples of the multi-well plate may include 24-well, 384-well, and 1536-well plates.

A 1 mL syringe is used to apply downward pressure on each glass rod, ensuring that all rods are in contact with the bottom of the multi-well plate (e.g., 96-well plate). Binders such as rubber bands are then used to secure the hydrogel stamp device to the multi-well plate, holding all components in position. In some implementations, an epoxy is applied to each glass rod, ensuring a secure attachment. The assembly is then covered and left to dry, e.g., for a predetermined period of time (e.g., a period of 24 hours).

Surface Treatment of the Hydrogel Stamp Device with Siliconizing Agent

Once the epoxy has fully cured, the polished ends of the glass rods undergo a surface treatment process using a siliconizing agent (e.g., Sigmacote) to enhance their hydrophobic properties. In some implementations, the glass rods are briefly flame-treated to remove any residual moisture, while ensuring that the plastic base does not melt during this process. After allowing the rods to cool for a predetermined period of time (e.g., approximately three minutes), the hydrogel stamp device is placed into a large plastic petri dish containing the siliconizing agent. The hydrogel stamp device is positioned such that the polished ends of the glass rods remain above the bottom of the petri dish while being in direct contact with the siliconizing agent. The hydrogel stamp device remains in this solution for a predetermined period of time (e.g., at least 30 minutes).

After the surface treatment process using the siliconizing agent (e.g., Sigmacote), a hydrophobic silicone layer is formed on the ends of the glass rods.

Following treatment, the stamp is removed from the siliconizing agent and washed, e.g., three times with deionized water, ensuring each wash lasts for a minimum of 15 minutes. The polished ends of the glass rods are then visually inspected to confirm their hydrophobicity. When exposed to water, the treated surfaces should cause water droplets to bead up rather than spread across the glass. If the hydrophobicity is insufficient, the siliconizing agent treatment process is repeated with a fresh batch of the solution.

After confirming proper surface treatment, the 0 mm spacer is removed, and the desired spacer is attached to the hydrogel stamp device to determine the final hydrogel height. In some implementations, the thickness of the spacer is determined based on (1) the height at which the glass rod protrudes above the base, (2) the depth of the well in the well plate, and (3) the desired thickness of the hydrogel. For example, a 0.25 mm spacer may be used for forming polyethylene glycol-norbornene (PEG-nb) hydrogels.

### Preparation of PEG-Nb Hydrogels on a Multi-Well Glass Bottom Plate

The disclosed technology can be implemented in some embodiments to provide devices and methods for preparing poly(ethylene glycol)-norbornene (PEG-nb) hydrogels for cell culture applications, particularly for seeding valvular interstitial cells (VICs).

The hydrogel preparation process based on some embodiments may include a vapor deposition process, a hydrogel precursor preparation process, and a curing process.

In some embodiments of the disclosed technology, vapor deposition materials and equipment include a multi-well glass-bottom plate (e.g., 96-well glass-bottom plate), a scintillation vial, an autoclave container (e.g., autoclave jar), and mercaptopropyltrimethoxysilane (MPTS).

In some embodiments of the disclosed technology, PEG-nb gel preparation materials and equipment include phosphate-buffered saline (PBS), PEG-dithiol (PEG-diSH), PEG-norbornene (PEG-nb), arginylglycylaspartic acid (RGD), lithium phenyl-2,4,6-trimethylbenzoylphosphinate (LAP), a 5% isopropyl alcohol (IPA) solution in PBS, 1% fetal bovine serum (FBS) VIC media, multi-well plate stamp (e.g., 96-well plate stamp), and multi-channel pipet.

### Vapor Deposition Process on Multi-Well Glass-Bottom Plates

The hydrogel preparation process based on some embodiments may include a vapor deposition process that includes performing vapor deposition on a multi-well glass-bottom plate (e.g., 96-well glass-bottom plate). In some implementations, this process begins by heating an oven to 60° C. The vapor deposition materials and equipment discussed above are gathered within a chemical fume hood, including a scintillation vial, an autoclave jar, and mercaptopropyltrimethoxysilane (MPTS). In some implementations, a volume of 100 μL of MPTS is transferred into the scintillation vial, which is then left uncapped and placed inside the autoclave jar along with the multi-well glass-bottom plate. The lid of the well plate is kept inside the fume hood to avoid contamination.

The autoclave jar is loosely closed and placed into the oven, where it remains for a minimum of six hours. Once the vapor deposition process is complete, the jar is removed from the oven and opened within the fume hood. The scintillation vial is rinsed with acetone. The lid of the 96-well plate is then labeled to indicate that vapor deposition has been performed. The multi-well plate is covered with the lid and stored until it is ready for hydrogel attachment, with storage permissible for several days.

### Preparation of Gel Precursor Solution and Pipet Using Liquid Handling System

The hydrogel preparation process based on some embodiments may include a hydrogel precursor preparation process. The hydrogel precursor solution is transferred to a liquid handling system (e.g., EpMotion liquid handling system) for automated dispensing, using software (e.g., epBlue) to load the hydrogel protocol, ensuring that the multi-well plate and gel solution are correctly positioned within the liquid handling system.

The software (e.g., epBlue) is launched, and both the well plate and gel solution are added to the liquid handling system (e.g., EpMotion).

Before proceeding, the liquid handling system (e.g., EpMotion) can be configured as follows. For example, the user logs into the software (e.g., epBlue), navigates to settings, and selects devices. In the available devices section, the desired system (e.g., 5073KQ707256) is selected. In some implementations, to ensure a sterile environment, the clean cap UV light/HEPA filter option is chosen, and the start button is clicked under HEPA filter activation. The dispensing tools is removed before activating the UV light, and after sterilization, they are wiped down with 70% ethanol before being placed back into the liquid handling system (e.g., EpMotion).

The hydrogel precursor solution may be prepared within a biosafety cabinet. In some implementations, a volume of approximately 7 μL of hydrogel precursor solution is allocated per gel, with 14 extra gels per plate to account for dead volume.

In some implementations, within the software, an application editor is opened, and a hydrogel protocol is selected. If any discrepancies are found, the workable settings in the software are adjusted to match the workable settings in the liquid handling system.

Before running the protocol, the vapor deposition-treated 96-well plate is thoroughly sprayed with ethanol and placed in the correct location according to the worktable settings, ensuring that the lid is removed. Similarly, an Eppendorf tube containing the hydrogel precursor solution is also sprayed with ethanol and placed in the correct position. If the correct placement of the hydrogel Eppendorf tube is unclear, the mix or sample transfer step within the software is selected, followed by the pattern option, to verify the correct location.

Once all components are correctly positioned, the cover of the liquid handling system (e.g., EpMotion) is closed, and the procedure is initiated by clicking the play button. The system will prompt the user to click through a series of “next” buttons, which should be completed as they appear. The HEPA filter remains running throughout the procedure. In some implementations, when prompted, “1” is entered for the number of samples, and the run button is clicked to begin dispensing.

The liquid handling system (e.g., EpMotion) then automatically dispenses the hydrogel precursor solution into each well. Once the dispensing process is completed, the well plate is carefully removed and covered with the lid. To ensure uniform gel formation, the well plate remains flat and steady until it is exposed to UV light, thereby preventing the gel precursor solution from shifting or adhering to the side of the wells.

### Hydrogel Formation and Wash Steps

The hydrogel preparation process based on some embodiments may include forming the hydrogels under UV light. The well plate is carefully transported to the tissue culture room, where it is sprayed with ethanol and placed inside the UV biosafety cabinet. The hydrogel stamp device (e.g., 96-well stamp) is also sterilized by spraying it with ethanol and patting it dry using a disposable wipe (e.g., Kimwipe). To further ensure a clean surface, the stamp is then sprayed with 5% isopropyl alcohol (IPA) and dried with a disposable wipe (e.g., Kimwipe). This step is repeated once more to ensure complete sterilization.

The hydrogel precursor solution is then stamped by gently pressing the hydrogel stamp device onto the multi-well plate. While maintaining pressure, the entire assembly of the multi-well plate and the hydrogel stamp device is flipped over so that the glass bottom of the multi-well plate is facing upward. While keeping the hydrogel stamp device firmly in place, the entire assembly of the multi-well plate and the hydrogel stamp device is positioned under the UV light source, where the hydrogels are cured for three minutes.

After curing, the stamp is carefully removed, and the hydrogels undergo a sterilization and wash process. The well plate is flipped back to its original orientation, and the stamp is gently pulled away to prevent any damage to the gels. Each well is then sterilized by adding approximately 400 μL of 5% IPA in PBS and allowing it to sit for at least 20 minutes. During this time, 1% FBS media is added to the water bath to pre-warm it. Additionally, the lid of the 96-well plate is wiped down with 5% IPA solution to ensure sterility.

Following sterilization, the wells are washed twice using 200 μL of PBS per well to remove any residual IPA. The gels are then prepared for cell culture by swelling in 1% FBS VIC media. The PBS is removed from each well, and 150 μL of warmed 1% FBS VIC media is added to each well. The multi-well plate is then placed into an incubator and maintained under appropriate conditions until the experiment is ready to be performed.

After curing, the well plate is returned to its original orientation, and the hydrogel stamp device is carefully removed. The wells are then sterilized by adding approximately 400 μL of a 5% IPA solution in PBS per well, allowing for at least 20 minutes of sterilization. The wells are subsequently washed twice with PBS at a volume of 200 μL per wash. Finally, the hydrogels are left to swell in 1% FBS VIC media by removing the PBS and adding 150 μL of the warmed media to each well. The well plate is then placed into an incubator until the hydrogels are ready for experimentation.

In this way, the high-throughput hydrogel preparation system can efficiently generate uniform, flat hydrogels suitable for various biological and chemical applications.

The disclosed technology can be implemented in some embodiments to provide a system for increasing the throughput of experiments requiring softer substrates, allowing for a greater number of conditions to be tested per experiment. The system utilizes a hydrogel preparation and stamping method optimized for a multi-well plate (e.g., 96-well plate).

As will be discussed below, to achieve reproducible and high-throughput hydrogel formation, various modifications and optimizations were made, including surface treatments, hydrogel volume adjustments, and material selections for the stamp.

### Optimization of Thiolation and Substrate Preparation

To improve hydrogel adhesion and ensure compatibility with soft substrates, the 96-well glass-bottom plate undergoes plasma cleaning for one minute prior to thiolation. The thiolation solution consists of 3.75 mL of toluene, 562.5 μL of mercaptopropyltrimethoxysilane (MPTS), and 187.5 μL of 2-butylamine, totaling 4.5 mL. However, initial attempts with this solution resulted in plastic melting. To address this issue, vapor deposition thiolation was tested as an alternative method. In this approach, 100 μL of MPTS was placed into a vial inside an autoclave jar containing the well plate. The autoclave jar was then placed in a vacuum oven at 10 inHg overnight at 60° C., ensuring that the jar lid was not fully screwed on to allow for proper vapor diffusion.

### Hydrogel Preparation and Volume Optimization

To determine the optimal hydrogel volume, multiple trials were conducted using both soft (4%) and stiff (10%) hydrogels. Hydrogels were polymerized under UV light for three minutes and then treated with 40 μL of phosphate-buffered saline (PBS). Different hydrogel volumes were tested across column 1 of the well plate, with volumes of 3, 4, 5, and 6 μL for the 4% gels and 3, 4, 5, and 6 μL for the 10% gels.

It was observed that the hydrogels adhered well to the well plate, allowing for further optimization of the high-throughput design. The well dimensions were measured to ensure compatibility with the hydrogel stamping process. The inner well diameter measured 6.23 mm, while the outer diameter at the top was 8 mm. The well diameter at the bottom was approximately 5.9 mm, with an average center-to-center distance of 9 mm. Depth measurements across wells ranged from 11.28 mm to 11.36 mm.

To estimate gel thickness, dried gels were measured using calipers, and their height was calculated using the volume equation:

$$H = 4 V / ( π {D}^{2} ) \qquad ( Eq . 1 )$$

Here, “H” represents the height of the gel, “V” represents the volume of the gel, and “D” represents the diameter of the gel.

For different hydrogel volumes, the corresponding heights were estimated as follows:

TABLE 1

| Gel Volume (uL) | Gel Diameter (mm) | Height (um) |
|---|---|---|
| 3 | 2.33 | 704 |
| 4 | 3.12 | 523 |
| 6 | 4.3 | 413 |
| 3 | 2.78 | 494 |
| 4 | 3.65 | 382 |
| 5 | 4.28 | 348 |
| 6 | 5.84 | 224 |

In the experiment shown in Table 1, a 3 μL gel with a 2.33 mm diameter resulted in a height of approximately 704 μm, a 4 μL gel with a 3.12 mm diameter resulted in a height of 523 μm, a 6 μL gel with a 4.3 mm diameter resulted in a height of 413 μm, a 3 μL gel with a 2.78 mm diameter resulted in a height of 494 μm, a 4 μL gel with a 3.65 mm diameter resulted in a height of 382 μm, a 5 μL gel with a 4.28 mm diameter resulted in a height of 348 μm. A 6 μL gel with a 5.84 mm diameter resulted in a height of 224 μm. However, this sample contained bubbles, so the measurement may be inaccurate.

In the experiment discussed above, gel thicknesses generally ranged between 300 and 500 μm. Additional trials using a 3D-printed gel stamp were conducted, forming seven hydrogels per column at 5 μL volumes in both the 4% and 10% conditions. Some gels adhered to the plate, while others remained attached to the stamp, indicating a need for a more hydrophobic gel stamp material.

### Optimization of the Gel Stamping Process

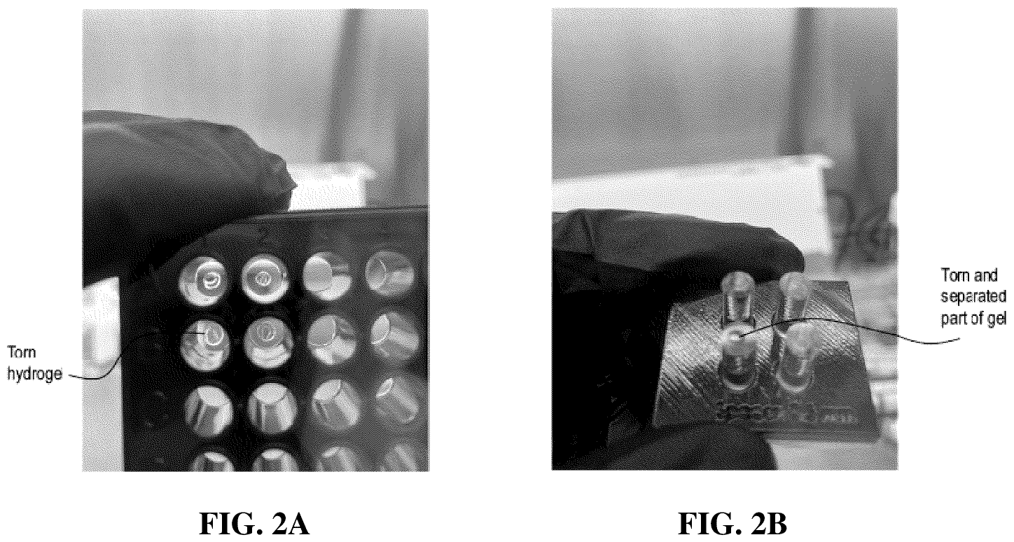

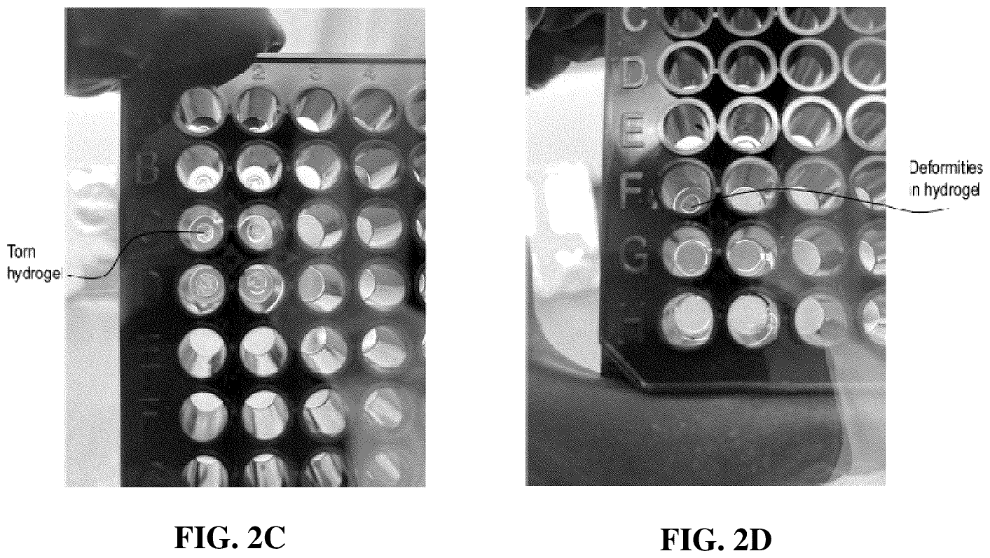

FIGS. 2A-2D shows the results of experiments conducted for optimization of the gel stamping process.

To refine hydrogel stamping and improve adherence, multiple attempts were made using different gel volumes and stamp modifications.

Referring to FIGS. 2A-2B, in the first attempt, soft hydrogels at 3 μL and 4 μL volumes were tested using a 0.25 mm spacer and the base stamp. While some gels adhered to the glass, the gel centers tore when the stamp was removed. The 3 μL gels were determined to be too small, while the 4 μL gels produced better but still suboptimal results.

Referring to FIG. 2C, the second attempt involved testing soft hydrogels at 5 μL and 6 μL volumes using the same spacer and a stamp with hydrogel solution. While these gels better fit the stamp size, they still adhered to the stamp and tore in the middle. The 6 μL gels produced the largest and flattest hydrogels.

Referring to FIG. 2D, in the third attempt, stiff gels at 6 μL and 7 μL volumes were tested using a Sigmacote-treated stamp. Although this treatment prevented gel adherence to the stamp, some deformities in the hydrogels were observed. A smoother stamp surface was identified as a requirement for better results. The 7 μL volume provided nearly full well coverage, but inconsistencies in stamping pressure resulted in uneven gels.

The hydrogel adhesion in a thiolated 96-well plate was tested after swelling in PBS over a weekend.

To form the hydrogels, a gel precursor solution was prepared, and a 96-well glass bottom plate was obtained. A predetermined volume of the precursor solution was pipetted onto a Sigmacote-treated stamp, which was then pressed onto the surface of the 96-well plate. Photopolymerization was performed for approximately three minutes under ultraviolet (UV) light. The stamp was held in place, and the well plate was flipped over to facilitate removal of the stamp while ensuring that the gels adhered to the plate. The gels were subsequently cured, and each well was swelled with 150 μL of PBS before incubation over the weekend.

Observations indicated that, after the first stamping process, some gels adhered to the stamp rather than the well plate. This issue was resolved by wiping each glass rod with an ethanol-soaked wipe (e.g., Kimwipe) after each use. It was further observed that 8-μL gels did not consistently form a round shape (two of four 8-μL gels did not form a round gel), whereas 6-μL and 7-μL gels, formed using 0.25 mm spacers, demonstrated optimal morphology.

After three days of incubation in PBS, all gels remained adhered to the well plate, with 6-μL and 7-μL gels exhibiting minor folds. Gels seemed to spread out a little more and small wrinkles in gels became more apparent. This may be caused by tears formed when stamping the gel. The 4 μL and 5 uL gels looked the flattest after 3 days. 6 μL and 7 uL gels had noticeable folds.

After five days of incubation, all gels remained adhered. Most gels appeared to continue spreading across the bottom of the well, which was potentially due to residual PBS that could not be pipetted out.

After ten days of incubation, all gels remained adhered. Upon drying, the gels had spread slightly, but this spreading was minimal.

### Materials Used in Hydrogel Formation

The materials used for hydrogel formation included: 96 glass rods, with a 5-mm diameter, 21-mm length, and one end mechanically polished to a square shape within +5 degrees; 96-well glass bottom plate; 3D-printed base obtained; four spacers (0 mm, 0.25 mm, 0.5 mm, and 1 mm); epoxy for securing glass rods; and sticks for applying epoxy.

### Stamp Preparation and Sigmacote Treatment

To prepare the stamp, all 96 glass rods were placed into the 3D-printed base, ensuring that the polished ends faced outward. A 0-mm spacer was positioned onto the base, and the stamp with the spacer was inserted into the 96-well glass bottom plate. A syringe was used to press each glass rod to ensure uniform contact with the bottom of the plate. Binders such as rubber bands were used to secure the stamp and spacer assembly. Epoxy was applied using a stick to glue each glass rod, and the assembly was left undisturbed for 24 hours.

To enhance the anti-adhesive properties of the stamp, a Sigmacote treatment was performed. In an implementation, a plasma treatment of the epoxy can be performed. In another implementation, a flame treatment can be performed. In this experiment, a flame treatment was employed. The stamp was flame-treated using large tweezers, ensuring that plastic components were not melted. The stamp was then placed in a large plastic petri dish containing Sigmacote for at least 30 minutes. The Sigmacote was subsequently recovered for reuse, and the stamp was rinsed three times with deionized water, drying with a Kimwipe between washes.

### Preparation of Soft and Stiff Hydrogels for Cell Seeding

Two days prior to the experiment, male and female porcine valvular interstitial cells (VICs) were thawed. One day before the experiment, soft and stiff hydrogels were prepared as follows.

The gel precursor solution was prepared by sequentially adding PBS, polyethylene glycol (PEG) dithiol, PEG norbornene (PEGnb), and cyclic Arg-Gly-Asp-Ser (cRGDS). The solution was handled in a dark environment, and an Eppendorf tube containing the solution was wrapped in foil. Lithium phenyl-2,4,6-trimethylbenzoylphosphinate (LAP) was then added.

The desired hydrogel volume was pipetted onto the Sigmacote-treated glass rod stamp, which had been previously wiped with ethanol. The 96-well glass bottom plate was carefully pressed onto the stamp, inverted, and photopolymerized for three minutes under UV light. The stamp was then carefully removed, and adherence of all hydrogels was confirmed. The hydrogels were sterilized by adding 100 μL of 5% isopropyl alcohol in PBS using a multichannel pipette. The wells were washed three times with PBS, followed by the addition of 150 μL of 1% fetal bovine serum (FBS) to each well. The hydrogels were then examined under a microscope, and all gels remained well-formed, with no adhesion to the stamp. However, 4-μL and 5-μL gels were found to be too small to adequately cover the bottom of the wells.

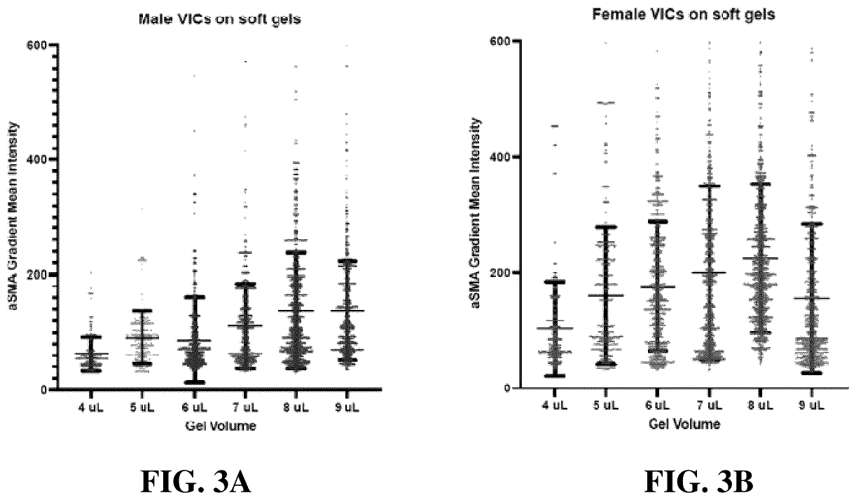

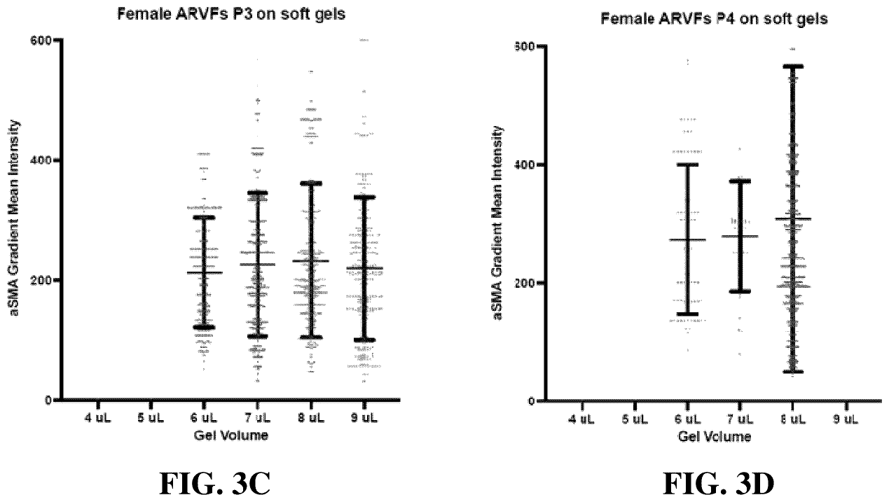

FIG. 3A shows αSMA (Alpha-Smooth Muscle Actin) gradient mean intensity of male VICs on soft gels. FIG. 3B shows αSMA gradient mean intensity of female VICs on soft gels. FIG. 3C shows αSMA gradient mean intensity of female adult rat ventricular fibroblasts (ARVFs) P3 (passage 3) on soft gels. FIG. 3D shows αSMA gradient mean intensity of female ARVFs P4 (passage 4) on soft gels.

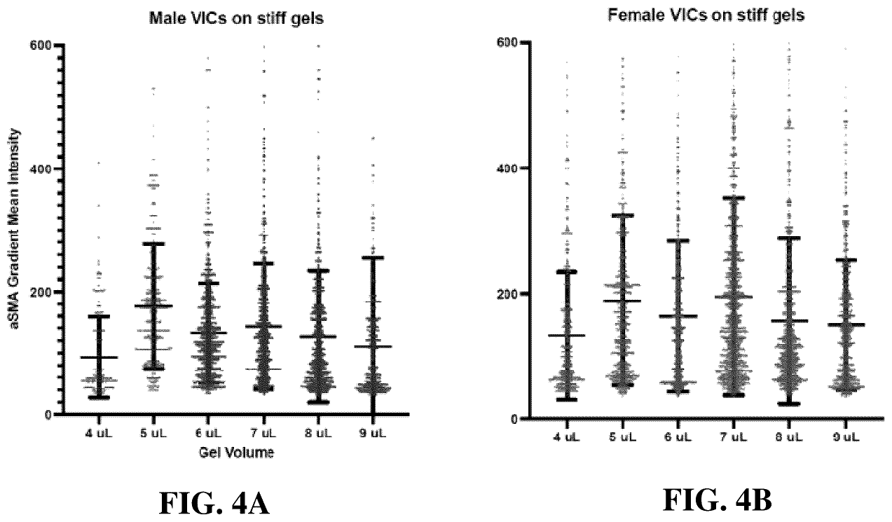

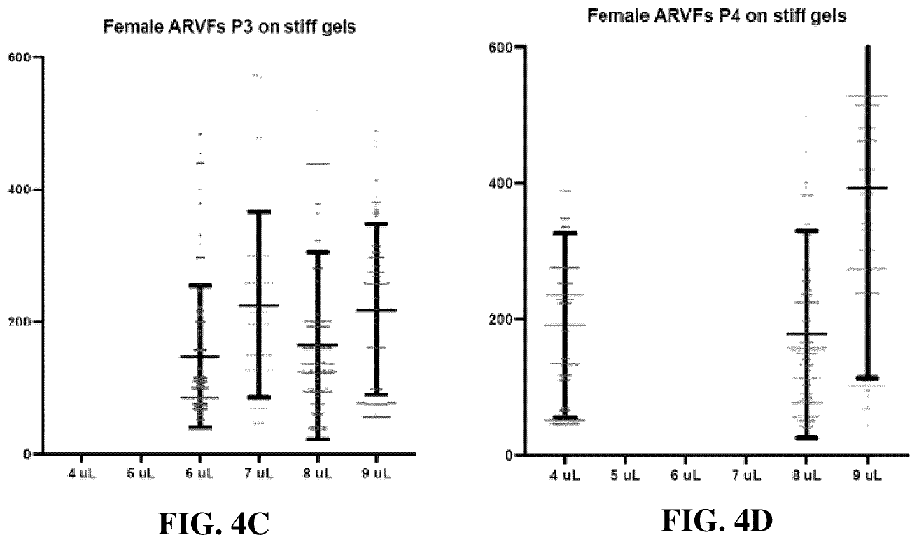

FIG. 4A shows αSMA gradient mean intensity of male VICs on stiff gels. FIG. 4B shows αSMA gradient mean intensity of female VICs on stiff gels. FIG. 4C shows αSMA gradient mean intensity of female ARVFs P3 on stiff gels. FIG. 4D shows αSMA gradient mean intensity of female ARVFs P4 on stiff gels.

On the day of the experiment, cells are trypsinized, and a portion of the VICs obtained from the initial isolation process, along with P3 and P4 adult rat ventricular fibroblasts (ARVFs), were prepared for seeding. The number of cells per tube was calculated as follows:

For male VICs: 6400 cells/150 μL×4000 μL=170,667 cells

For female VICs: 6400 cells/150 μL×4000 μL=170,667 cells

For ARVFs: 3200 cells/150 μL×4000 μL=85,333 cells

The volumes of cell solution added per tube were as follows:

Male VICs: 36.9 μL

Female VICs: 47.3 μL

ARVF P3: 194 μL

ARVF P4: 44 μL

The seeded hydrogels were incubated for 48 hours before proceeding to fixation, permeabilization, blocking, and immunostaining following established protocols. Samples were subsequently stored in PBS, wrapped in parafilm, and covered with foil for protection. Results were shown in FIGS. 3A-3D and 4A-4D.

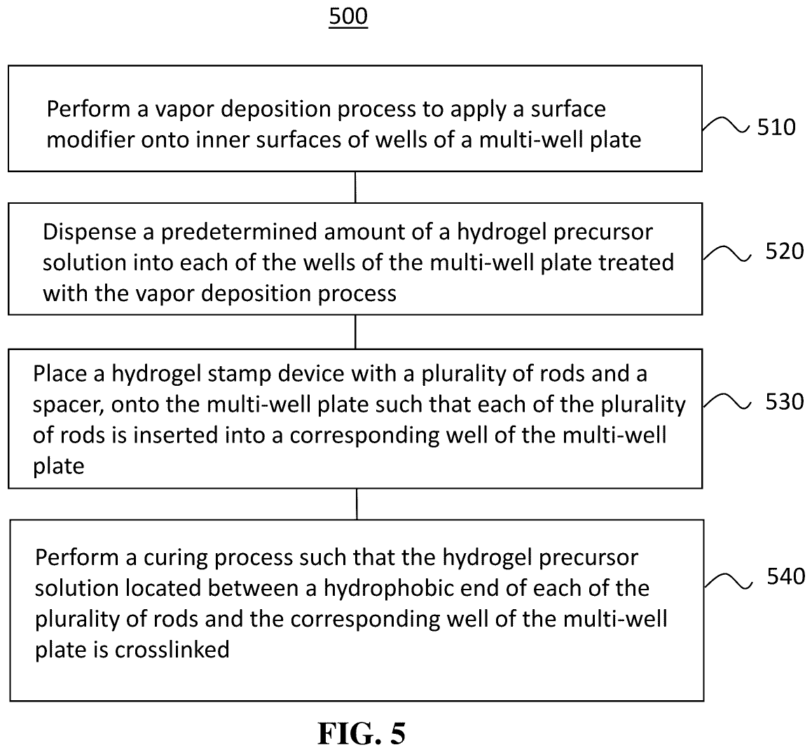

FIG. 5 shows an example method 500 of simultaneously producing a plurality of hydrogels based on some embodiments of the disclosed technology.

In some implementations, the method 500 may include, at 510, performing a vapor deposition process to apply a surface modifier onto inner surfaces of wells of a multi-well plate, at 520, dispensing a predetermined amount of a hydrogel precursor solution into each of the wells of the multi-well plate treated with the vapor deposition process, at 530, placing a hydrogel stamp device with a plurality of rods and a spacer, onto the multi-well plate such that each of the plurality of rods is inserted into a corresponding well of the multi-well plate, and at 540, performing a curing process such that the hydrogel precursor solution located between a hydrophobic end of each of the plurality of rods and the corresponding well of the multi-well plate is crosslinked. For example, the curing process may be performed by exposure to light of a specific wavelength such as ultra-violet light (UV), visible light, and infrared light (IR). Aa another example, the curing process may include chemical crosslinking, thermal crosslinking, self-crosslinking, and others.

Therefore, various implementations of features of the disclosed technology can be made based on the above disclosure, including the examples listed below.

Example 1. A hydrogel stamp device, comprising: a base; a plurality of rods arranged on the base and configured to be inserted into a plurality of wells of a multi-well plate to enable formation of hydrogels inside the plurality of wells, wherein each of the plurality of rods extends vertically from the base and includes: a base end connected to the base; a hydrophobic end positioned opposite the base end; and a body portion between the base end and the hydrophobic end; and a spacer that surrounds at least part of the base ends of the plurality of rods.

Example 2. The device of example 1, wherein the plurality of rods includes glass rods.

Example 3. The device of example 1, wherein each of the plurality of rods includes a hydrophobic silicone layer on the hydrophobic end.

Example 4. The device of example 1, wherein the spacer is detachable from the base.

Example 5. The device of example 1, wherein the spacer includes a plurality of holes to allow the plurality of rods to be inserted and pass therethrough such that the spacer is placed on the base to surround the base end of each of the plurality of rods.

Example 6. The device of example 5, wherein a number of the plurality of holes is identical to a number of the plurality of rods.

Example 7. The device of example 1, wherein a thickness of the spacer is based on: a height at which the plurality of rods protrudes above the base; a depth of the plurality of wells in the multi-well plate, and a thickness of the hydrogels.

Example 8. The device of example 1, wherein each of the plurality of rods has a same two-dimensional shape as a corresponding well of the multi-well plate.

Example 9. The device of example 1, wherein each of the plurality of rods is transparent to ultraviolet light.

Example 10. A method of simultaneously forming a plurality of hydrogels, comprising: performing a vapor deposition process to apply a surface modifier onto inner surfaces of wells of a multi-well plate; dispensing a predetermined amount of a hydrogel precursor solution into each of the wells of the multi-well plate treated with the vapor deposition process; placing a hydrogel stamp device with a plurality of rods and a spacer, onto the multi-well plate such that each of the plurality of rods is inserted into a corresponding well of the multi-well plate; and performing a curing process such that the hydrogel precursor solution located between a hydrophobic end of each of the plurality of rods and the corresponding well of the multi-well plate is crosslinked.

Example 11. The method of example 10, wherein the surface modifier includes mercaptopropyltrimethoxysilane (MPTS).

Example 12. The method of example 10, further comprising performing a surface treatment process on the hydrophobic end of each of the plurality of rods using a siliconizing agent.

Example 13. The method of example 10, wherein the hydrogel precursor solution includes at least one of phosphate-buffered saline (PBS), polyethylene glycol (PEG) dithiol, PEG norbornene (PEGnb), cyclic Arg-Gly-Asp-Ser (cRGDS), or lithium phenyl-2,4,6-trimethylbenzoylphosphinate (LAP).

Example 14. The method of example 13, wherein the hydrogel precursor solution is formed by sequentially adding the PBS, the PEG dithiol, the PEGnb, and the cRGDS in a dark environment, and adding LAP.

Example 15. The method of example 10, further comprising replacing the spacer with another spacer of a different thickness to adjust a thickness of the plurality of hydrogels.

Example 16. The method of example 10, further comprising adding an additional spacer to adjust a thickness of the plurality of hydrogels.

Example 17. The method of example 10, wherein a bottom of the multi-well plate is transparent to ultraviolet light.

Example 18. The method of example 10, wherein the hydrophobic end of each of the plurality of rods is transparent to ultraviolet light.

Example 19. The method of example 10, wherein the plurality of hydrogels include poly(ethylene glycol)-norbornene (PEG-nb) hydrogel for cell culture applications.

Example 20. The method of example 10, wherein the plurality of hydrogels include hydrogels for seeding valvular interstitial cells (VICs).

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A hydrogel stamp device, comprising:
a base;
a plurality of rods arranged on the base and configured to be inserted into a plurality of wells of a multi-well plate to enable formation of hydrogels inside the plurality of wells, wherein each of the plurality of rods extends vertically from the base and includes:
a base end connected to the base;
a hydrophobic end positioned opposite the base end; and
a body portion between the base end and the hydrophobic end; and
a spacer that surrounds at least part of the base ends of the plurality of rods.

2. The device of claim 1, wherein the plurality of rods includes glass rods.

3. The device of claim 1, wherein each of the plurality of rods includes a hydrophobic silicone layer on the hydrophobic end.

4. The device of claim 1, wherein the spacer is detachable from the base.

5. The device of claim 1, wherein the spacer includes a plurality of holes to allow the plurality of rods to be inserted and pass therethrough such that the spacer is placed on the base to surround the base end of each of the plurality of rods.

6. The device of claim 5, wherein a number of the plurality of holes is identical to a number of the plurality of rods.

7. The device of claim 1, wherein a thickness of the spacer is based on: a height at which the plurality of rods protrudes above the base; a depth of the plurality of wells in the multi-well plate; and a thickness of the hydrogels.

8. The device of claim 1, wherein each of the plurality of rods has a same two-dimensional shape as a corresponding well of the multi-well plate.

9. The device of claim 1, wherein each of the plurality of rods is transparent to ultraviolet light.

10. A method of simultaneously forming a plurality of hydrogels, comprising:
performing a vapor deposition process to apply a surface modifier onto inner surfaces of wells of a multi-well plate;
dispensing a predetermined amount of a hydrogel precursor solution into each of the wells of the multi-well plate treated with the vapor deposition process;
placing a hydrogel stamp device with a plurality of rods and a spacer, onto the multi-well plate such that each of the plurality of rods is inserted into a corresponding well of the multi-well plate; and
performing a curing process such that the hydrogel precursor solution located between a hydrophobic end of each of the plurality of rods and the corresponding well of the multi-well plate is crosslinked.

11. The method of claim 10, wherein the surface modifier includes mercaptopropyltrimethoxysilane (MPTS).

12. The method of claim 10, further comprising performing a surface treatment process on the hydrophobic end of each of the plurality of rods using a siliconizing agent.

13. The method of claim 10, wherein the hydrogel precursor solution includes at least one of phosphate-buffered saline (PBS), polyethylene glycol (PEG) dithiol, PEG norbornene (PEGnb), cyclic Arg-Gly-Asp-Ser (cRGDS), or lithium phenyl-2,4,6-trimethylbenzoylphosphinate (LAP).

14. The method of claim 13, wherein the hydrogel precursor solution is formed by sequentially adding the PBS, the PEG dithiol, the PEGnb, and the cRGDS in a dark environment, and adding LAP.

15. The method of claim 10, further comprising replacing the spacer with another spacer of a different thickness to adjust a thickness of the plurality of hydrogels.

16. The method of claim 10, further comprising adding an additional spacer to adjust a thickness of the plurality of hydrogels.

17. The method of claim 10, wherein a bottom of the multi-well plate is transparent to ultraviolet light.

18. The method of claim 10, wherein the hydrophobic end of each of the plurality of rods is transparent to ultraviolet light.

19. The method of claim 10, wherein the plurality of hydrogels include poly(ethylene glycol)-norbornene (PEG-nb) hydrogel for cell culture applications.

20. The method of claim 10, wherein the plurality of hydrogels include hydrogels for seeding valvular interstitial cells (VICs).
